(19) United States
(12) Patent Application Publication
(10) Pub. No.: US 2023/0123551 A1
(43) Pub. Date: Apr. 20, 2023

(54) VEHICLE STERILIZATION SYSTEM AND STERILIZATION METHOD

(71) Applicant: Gogoro Inc., Hong Kong (CN)

(72) Inventors: Hok-Sum Horace Luke, Mercer Island, WA (US); Chun-Jen Tsai, Taoyuan City (TW); Chih-Yuan Chen, Taoyuan City (TW); Ching-Chang Ni, Taoyuan City (TW); Wen-Hsien Tseng, Taoyuan City (TW)

(21) Appl. No.: 18/045,941

(22) Filed: Oct. 12, 2022

Related U.S. Application Data
(60) Provisional application No. 63/255,536, filed on Oct. 14, 2021.

(51) Int. Cl. A61L 2/26; A61L 2/10; B62J 9/14

(52) U.S. Cl. CPC A61L 2/26; A61L 2/10; B62J 9/14

(57) ABSTRACT

A sterilization method for a vehicle sterilization system, comprising: receiving a detection signal, and determining whether a seat cushion of a vehicle covers a space of a compartment according to a voltage level of the detection signal; when the seat cushion does not completely cover the space of the compartment, controlling a power supply unit to stop supplying power; when the seat cushion covers the space of the compartment, controlling the power supply unit to supply power to a sterilization device.

### CROSS-REFERENCE TO RELATED APPLICATION

This application claims priority to U.S. Provisional Application Ser. No. 63/255,536, filed Oct. 14, 2021, which is herein incorporated by reference in its entirety.

### BACKGROUND

### Technical Field

The present disclosure relates to a vehicle sterilization system and a sterilization method, especially a technology for sterilizing a compartment by using a photocatalyst element.

### Description of Related Art

In recent years, people have paid more and more attention to environmental hygiene, and various antibacterial products have been developed accordingly. Vehicles such as cars or motorcycles carry different passengers and may pass through high-risk areas (also known as epidemic hot zones) such as hospitals and stations, so there is a higher demand for sterilization.

### SUMMARY

One aspect of the present disclosure is a sterilization method for a vehicle sterilization system, comprising: receiving a detection signal, and determining whether a seat cushion of a vehicle covers a space of a compartment according to a voltage level of the detection signal; when the seat cushion does not completely cover the space of the compartment, controlling a power supply unit to stop supplying power; and when the seat cushion covers the space of the compartment, controlling the power supply unit to supply power to a sterilization device.

In one embodiment, controlling the power supply unit to supply power to the sterilization device comprises: driving the sterilization device for a predetermined time, and then controlling the power supply unit to stop supplying power to the sterilization device.

In one embodiment, the sterilization method further comprises: when the vehicle stops driving at least one wheel, converting, by an electrical energy recovery device, thermal energy or kinetic energy generated by the at least one wheel into recovered electrical energy; and providing at least one portion of the recovered electrical energy to the sterilization device.

In one embodiment, providing the at least one portion of the recovered electrical energy to the sterilization device comprises: providing a first portion of the recovered electrical energy to the sterilization device; and using a second portion of the recovered electrical energy to charge the power supply unit.

In one embodiment, the sterilization method further comprises: determining a residual power ratio in the power supply unit; and when the residual power ratio is less than a warning threshold, stopping supplying the recovered electric energy to the sterilization device, and using the recovered electric energy to charge the power supply unit.

In one embodiment, the sterilization method further comprises: determining, by a sensor, a concentration of a volatile organic compound in the compartment; and when the concentration of the volatile organic compound is greater than a detection threshold, controlling the power supply unit to supply power to the sterilization device for at least a predetermined time.

In one embodiment, the sterilization device comprises a first ultraviolet light source and a second ultraviolet light source, and the sterilization method further comprises: determining a distance between the vehicle and a portable electronic device, wherein the vehicle is communicatively connected to the portable electronic device; when the distance is greater than a distance threshold, controlling the power supply unit to supply power to the first ultraviolet light source and the second ultraviolet light source; and when the distance is less than or equal to the distance threshold, controlling the power supply unit to supply power to one of the first ultraviolet light source and the second ultraviolet light source.

In one embodiment, the sterilization device comprises a first ultraviolet light source and a second ultraviolet light source, and the sterilization method further comprises: obtaining, by a positioning device, a coordinate of the vehicle; connecting, by the vehicle or a portable electronic device, to a cloud server, wherein the vehicle is communicatively connected to the portable electronic device; when determining the coordinate is located in a marked area recorded in the cloud server, controlling the power supply unit to supply power to the first ultraviolet light source and the second ultraviolet light source; when determining the coordinate is outside the marked area, controlling the power supply unit to supply power to one of the first ultraviolet light source and the second ultraviolet light source.

In one embodiment, the sterilization method further comprises: when the sterilization device is not powered by the power supply unit, driving the sterilization device by a battery, wherein the battery is different from the power supply unit.

Another aspect of the present disclosure is a vehicle sterilization system, comprising a power supply unit and a sterilization device. The power supply unit is arranged in a vehicle. A compartment of the vehicle is provided with a seat cushion. The sterilization device is arranged in the compartment. When the seat cushion does not completely cover a space of the compartment, the power supply unit is configured to stop supplying power. Wherein when the seat cushion covers the space of the compartment, the power supply unit is configured to supply power to the sterilization device.

In one embodiment, the vehicle sterilization system further comprises a controller. The controller is configured to control the power supply unit to drive the sterilization device for a predetermined time. After the predetermined time, the controller is configured to control the power supply unit to stop supplying power to the sterilization device.

In one embodiment, the vehicle sterilization system further comprises a controller and an electrical energy recovery device. The controller is configured to control the power supply unit to drive the sterilization device for a predetermined time. The electrical energy recovery device is configured to convert thermal energy or kinetic energy generated by the at least one wheel into recovered electrical energy when the vehicle stops driving at least one wheel. The controller is configured to provide at least one portion of the recovered electrical energy to the sterilization device.

In one embodiment, the controller is configured to provide a first portion of the recovered electrical energy to the sterilization device, and is configured to use a second portion of the recovered electrical energy to charge the power supply unit.

In one embodiment, the controller is configured to determine a residual power ratio in the power supply unit. When the residual power ratio is less than a warning threshold, the controller is configured to stop supplying the recovered electric energy to the sterilization device, and is configured to use the recovered electric energy to charge the power supply unit.

In one embodiment, the vehicle sterilization system further comprises a controller and a sensor. The controller is configured to control the power supply unit to drive the sterilization device for a predetermined time. The sensor is coupled to the controller, and is configured to determine a concentration of a volatile organic compound in the compartment. When the concentration of the volatile organic compound is greater than a detection threshold, the controller is configured to control the power supply unit to supply power to the sterilization device for at least a predetermined time.

In one embodiment, the sterilization device comprises at least one fluid generating element, at least one ultraviolet light source and at least one photocatalyst element, wherein a position of the at least one photocatalyst element corresponds to the at least one ultraviolet light source.

In one embodiment, the at least one ultraviolet light source comprises a first ultraviolet light source and a second ultraviolet light source, and the sterilization device further comprises a controller. The controller is configured to determine a distance between the vehicle and a portable electronic device. The vehicle is communicatively connected to the portable electronic device. When the distance is greater than a distance threshold, the controller is configured to control the power supply unit to supply power to the first ultraviolet light source and the second ultraviolet light source. When the distance is less than or equal to the distance threshold, the controller is configured to control the power supply unit to supply power to one of the first ultraviolet light source and the second ultraviolet light source.

In one embodiment, the at least one ultraviolet light source comprises a first ultraviolet light source and a second ultraviolet light source, and the sterilization device further comprises a controller and a positioning device. The controller is configured to control the power supply unit to drive the sterilization device for a predetermined time. The positioning device is configured to obtain a coordinate of the vehicle. When the coordinate is located in a marked area recorded in the cloud server, the controller is configured to control the power supply unit to supply power to the first ultraviolet light source and the second ultraviolet light source. When the coordinate is outside the marked area, the controller is configured to control the power supply unit to supply power to one of the first ultraviolet light source and the second ultraviolet light source.

In one embodiment, the sterilization device further comprises a lighting circuit. The lighting circuit is coupled to the power supply unit, and comprises a circuit board and at least one lighting element. The circuit board is configured to cover the at least one ultraviolet light source to prevent a ultraviolet light generated by the at least one ultraviolet light source from being exposed to an outside of the sterilization device.

In one embodiment, the vehicle sterilization system further comprises a controller and a battery. The controller is configured to control the power supply unit to drive the sterilization device for a predetermined time. The battery is coupled to the sterilization device. When the sterilization device is not powered by the power supply unit, the controller is configured to drive the sterilization device by the battery.

Accordingly, by arranging the sterilization device in the compartment, and confirming the state of the vehicle by an arrangement relationship between the seat cushion and the compartment, the sterilization device can automatically deodorize and sterilize the vehicle to ensure cleanliness of the vehicle and items in the compartment.

It is to be understood that both the foregoing general description and the following detailed description are by examples, and are intended to provide further explanation of the disclosure as claimed.

### BRIEF DESCRIPTION OF THE DRAWINGS

The present disclosure can be more fully understood by reading the following detailed description of the embodiment, with reference made to the accompanying drawings as follows:

FIG. 1A is a schematic diagram of a vehicle and a sterilization system in some embodiments of the present disclosure.

FIG. 1B is a schematic diagram of a vehicle and a sterilization system in some embodiments of the present disclosure.

FIG. 2 is a schematic diagram of a compartment and a space of the vehicle in some embodiments of the present disclosure.

FIG. 3 is a schematic diagram of a sterilization device in some embodiments of the present disclosure.

FIG. 4 is a flowchart illustrating a sterilization method in some embodiments of the present disclosure.

### DETAILED DESCRIPTION

For the embodiment below is described in detail with the accompanying drawings, embodiments are not provided to limit the scope of the present disclosure. Moreover, the operation of the described structure is not for limiting the order of implementation. Any device with equivalent functions that is produced from a structure formed by a recombination of elements is all covered by the scope of the present disclosure. Drawings are for the purpose of illustration only, and not plotted in accordance with the original size.

It will be understood that when an element is referred to as being “connected to” or “coupled to”, it can be directly connected or coupled to the other element or intervening elements may be present. In contrast, when an element to another element is referred to as being “directly connected” or “directly coupled,” there are no intervening elements present. As used herein, the term “and/or” includes an associated listed items or any and all combinations of more.

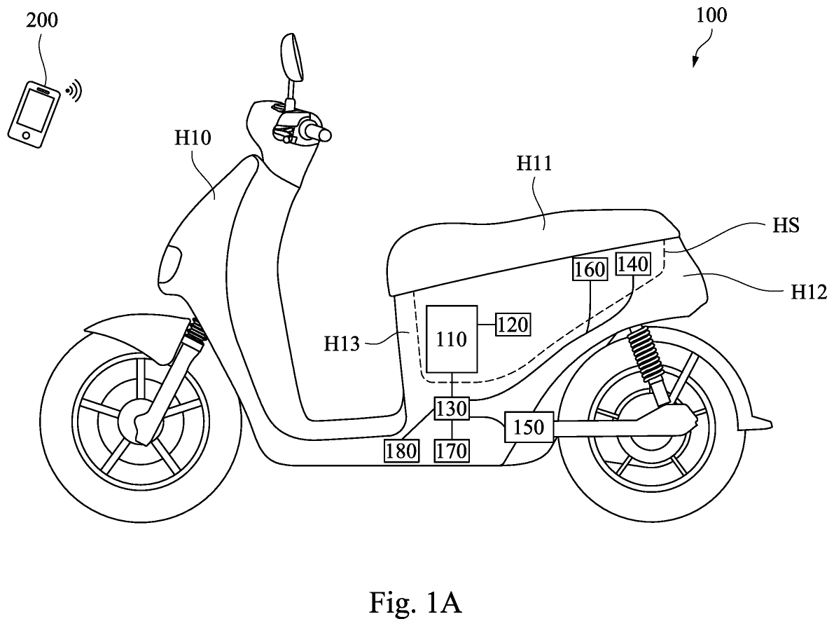

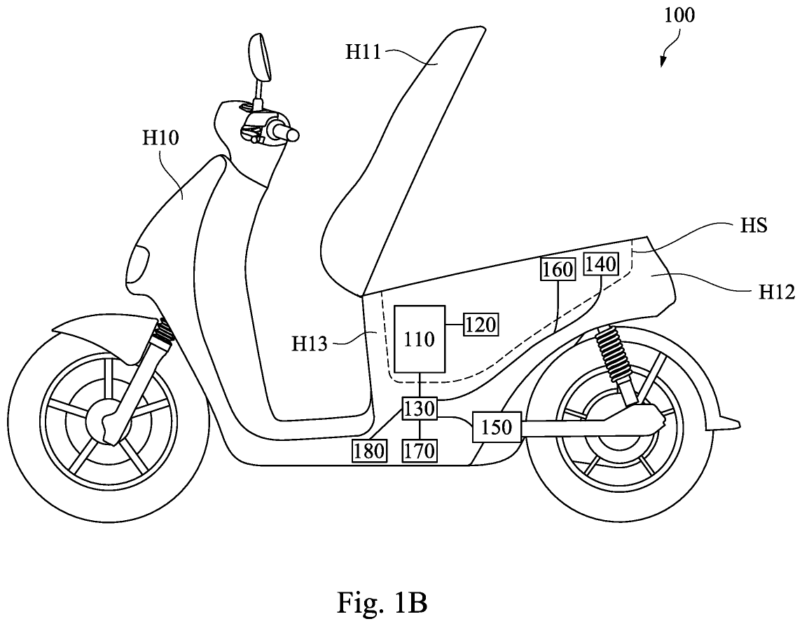

FIG. 1A and FIG. 1B are schematic diagrams of a vehicle H10 and a vehicle sterilization system 100 in some embodiments of the present disclosure. In one embodiment, the vehicle H10 applied by the vehicle sterilization system 100 is a two-wheeled motorcycle, and the vehicle sterilization system 100 is configured to sterilize a space HS in a compartment H12 under a seat cushion H11 of the vehicle H10. However, the present disclosure is not limited to this, in another embodiment, the vehicle sterilization system 100 can also sterilize other areas such as a windshield, a trunk (not shown in figure). In addition, the vehicle H10 can also be a three-wheeled motorcycle, an electric bicycle or other types of transportation.

The vehicle sterilization system 100 includes a power supply unit 110 and a sterilization device 120. The power supply unit 110 may be an energy storage device or a power supply circuit with the battery, which is pluggably arranged in the compartment H12 of the vehicle H10. The vehicle H10 may be implemented to an electric vehicle. The power supply unit 110 is configured to provide electrical energy to the vehicle H10, so as to drive a power system of the vehicle H10. However, the present disclosure is not limit to this. In other embodiment, the vehicle H10 may be implemented to a vehicle that uses gasoline as fuel. The compartment H12 is provided with a seat cushion H11. In some embodiments, the seat cushion H11 is pivotally connected to one end of the body H13 of the vehicle H10, so as to be closely arranged above the compartment H12. The seat cushion H11 covers the space HS in the compartment H12, or is turned away from the compartment H12, exposing the space HS in the compartment H12. It should be mentioned that positions of the components shown in FIG. 1A and FIG. 1B are for illustration only, and the position of the power supply unit 110 and the position of the sterilization device 120 can be adjusted according to requirements. For example, the power supply unit 110 may be located outside the compartment H12. In another embodiment, the power supply unit 110 can be a built-in battery that cannot be pulled out.

The sterilization device 120 is arranged at a position adjacent to the power supply unit 110 in the compartment H12, and is coupled to the power supply unit 110. An electrical conduction relationship between the sterilization device 120 and the power supply unit 110 is controlled by a detection signal, so that the sterilization device 120 can selectively receive power provided by the power supply unit 110 to be driven. The sterilization device 120 includes at least one fluid generating element (e.g., a fan or other element that can extract fluid from the outside), at least one ultraviolet light source and a photocatalyst element. The sterilization device 120 is configured to use the ultraviolet light source to illuminate the photocatalyst element to generate negative oxygen ions, hydroxide ions, hydrogen peroxide and other active substances with high oxidizing ability. The sterilization device 120 is configured to generate air convection by the fluid generating element to distribute the above active substances in the space HS to achieve the sterilization effect. Since those skilled in the art can understand the principle of photocatalyst sterilization, it will not be repeated here. The structure of the sterilization device 120 will be described in detail in the following paragraphs. In other embodiments, the sterilization device 120 may include a plurality of fluid generating elements to accelerate the dispersion of active substances and improve air circulation.

In addition, in this embodiment, the photocatalyst element is used in conjunction with the ultraviolet light source for sterilization. However, the present disclosure is not limited to this. In other embodiments, the sterilization device 120 may use other techniques for sterilization. For example, the sterilization device 120 may be implemented as an ultraviolet germicidal lamp or an ozone sterilizer. In other words, as long as a device has sterilization function, it can be used as the sterilization device 120.

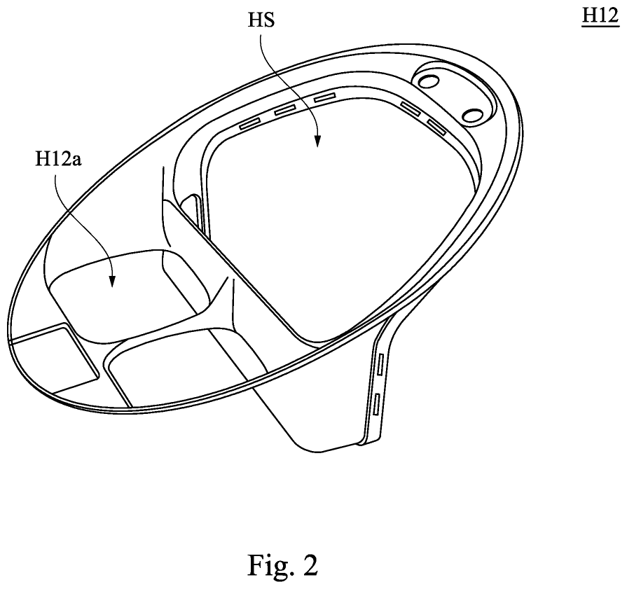

FIG. 2 is a schematic diagram of the compartment H12 and the space HS in some embodiments of the present disclosure. As shown in FIG. 1A, FIG. 1B and FIG. 2, in some embodiments, the power supply unit 110 includes one or more removable batteries (not shown in figure), and the removable batteries are arranged in battery slots H12a in the compartment H12 to respectively couple the vehicle H10 through electrodes in each battery. The top of the power supply unit 110 is exposed to the space HS, and an user can easily take out the power supply unit 110 to replace or repair the power supply unit 110. In another embodiment, the sterilization device 120 is arranged on a wall surface inside the compartment H12 (i.e., in the space HS), and at a position adjacent to the battery slot H12a. The power supply circuit of the vehicle H10 can be conveniently arranged around the battery slot H12a, so that the power supply unit 110 and the sterilization device 120 are coupled to each other. Alternatively, the sterilization device 120 is arranged elsewhere inside the compartment H12, which the present disclosure does not limit.

In some embodiments, the vehicle sterilization system 100 further includes a controller 130. The controller 130 is coupled to the power supply unit 110, so as to drive other electronic components of the vehicle H10 through electrical energy provided by the power supply unit 110. In some other embodiments, the controller 130 can be arranged in the power supply unit 110. In other words, the vehicle sterilization system 100 may include multiple controllers coupled to each other. For example, the vehicle H10 includes an electronic control unit (ECU) configured to drive other electronic components of the vehicle H10, and the power supply unit 110 includes a power supply controller.

In some embodiments, the vehicle sterilization system 100 further includes a state detection circuit 140. The state detection circuit 140 may be a sensor, and is configured to determine whether the seat cushion H11 completely covers the compartment H12 and completely covers the space HS in the compartment H12. For example, the state detection circuit 140 determines whether a rotation angle of the seat cushion H11 is within a predetermined range. If the rotation angle is within the predetermined range, it means that the seat cushion H11 has completely covered the space HS in the compartment H12.

In other embodiment, the state detection circuit 140 may be implemented to a switch circuit. When an U-shape hook at the bottom of the seat cushion H11 is inserted into a slot (e.g., buckle) in the compartment H12, the state detection circuit 140 will be turned on accordingly, that is, the state detection circuit 140 can determine that the seat cushion H11 is completely covered to the compartment H12.

As mentioned above, the state detection circuit 140 generates the corresponding detection signal according to the state of the seat cushion H11. As shown in FIG. 1A, when the seat cushion H11 completely covers the space HS in the compartment H12, the detection signal is at an enable level (e.g., low voltage). As shown in FIG. 1B, when the seat cushion H11 does not cover to the compartment H12, the detection signal is at a disable level (e.g., high voltage). Then, the state detection circuit 140 outputs the detection signal to the controller 130 or the power supply unit 110.

The vehicle sterilization system 100 automatically changes or controls whether the power supply unit 110 outputs electrical energy to the sterilization device 120 according to the detection signal (i.e., the state of the seat cushion H11). When the seat cushion H11 completely covers the space HS in the compartment H12, the controller 130 controls the power supply unit 110 to automatically output/supply power to the fluid generating element and the ultraviolet light source of the sterilization device 120 according to the detection signal at the enable level, so as to perform sterilization. On the other hand, if the seat cushion H11 does not completely cover the space HS in the compartment H12, the controller 130 controls the power supply unit 110 to stop supplying power to the sterilization device 120 according to the detection signal at the disable level. In short, the level of the detection signal can be automatically switched based on the state of the seat cushion H11, so that the controller 130 changes the electrical conduction relationship between the power supply unit 110 and the sterilization device 120. In another embodiment, the detection signal can be received by the power supply unit 110, and the power supply unit 110 automatically supplies power or stops supplying power to the sterilization device 120 according to the level of the detection signal.

In one embodiment, the controller 130 is configured to control the power supply unit 110 to provide electrical energy for a predetermined time (e.g., 10 minutes, 30 minutes or 60 minutes). After the predetermined time, the controller 130 controls the power supply unit 110 to stop supplying power to the fluid generating element and the ultraviolet light source of the sterilization device 120. For example, if the vehicle H10 is an electric motorcycle, when the user starts to ride the vehicle H10 (the seat cushion H11 must be closed to the compartment H12 at this time), the sterilization device 120 receives the power from the power supply unit 110, and performs at least one sterilization process. The time required for the sterilization process depends on a size of the compartment H12 and/or performance of the sterilization device 120. After finishing the sterilization process, the controller 130 (which can be the electronic control unit of the electric motorcycle, or the power supply controller of the power supply unit 110) will stop supplying power to the sterilization device 120.

In some embodiments, the vehicle sterilization system 100 further includes an electrical energy recovery device 150. The electrical energy recovery device 150 is coupled to the controller 130, and when the vehicle H10 stops providing power to at least one wheel (but the vehicle H10 is still coasting and the wheel continues rotating), the electrical energy recovery device 150 is configured to convert thermal energy and/or kinetic energy generated by the wheel into recovered electrical energy. The controller 130 is configured to provide at least one portion of the recovered electrical energy to the power supply unit 110 to drive the sterilization device 120, or is configured to directly supply the recovered electrical energy to the sterilization device 120. There are many ways and structures of the electrical energy recovery device 150. Since those skilled in the art can understand the operation principle of the electrical energy recovery device 150, it will not be repeated here.

In addition, in one embodiment, the controller 130 records how much the recovered electrical energy is generated by the electrical energy recovery device 150, and distributes the recovered electrical energy to the power supply unit 110 and the sterilization device 120, respectively. For example, when the user rides the vehicle H10, the controller 130 controls the power supply unit 110 to provide a first portion (e.g., 50%) of the recovered electrical energy to the sterilization device 120, and a second portion (e.g., another 50%) of the recovered electrical energy is retained in the power supply unit 110 to charge the power supply unit 110.

In other embodiment, the recovered electrical energy is provided directly to the sterilization device 120. For example, when the controller 130 detects that the electrical energy recovery device 150 generates the recovered electrical energy, the controller 130 directly inputs at least one portion of the recovered electrical energy to the sterilization device 120, and controls the power supply unit 110 to temporarily stop supplying power to the sterilization device 120. Accordingly, the sterilization device 120 may operate alone with the recovered electrical energy for a period of time until the recovered electrical energy is depleted.

The controller 130 dynamically controls the distribution of the recovered electrical energy. In one embodiment, the controller 130 determines the ratio (residual power ratio) of residual power in the power supply unit 110 to maximum capacity to determine how to distribute the recovered electrical energy. For example, if the controller 130 determines that the ratio of the residual power in the power supply unit 110 to the maximum capacity is less than a warning threshold (e.g., the current residual power is lower than 10%), the controller 130 stops providing the recovered electrical energy to the sterilization device 120, and charges the power supply unit 110 with all the recovered electrical energy.

In another embodiment, the vehicle sterilization system 100 determines whether to drive the sterilization device 120 according to a concentration of one or more specific gas molecules in the compartment H12. For example, the vehicle sterilization system 100 includes a sensor 160, and the sensor 160 is arranged in the compartment H12 or the space HS. The sensor 160 is coupled to the controller 130, is configured to detect a concentration of the volatile organic compounds (VOCs) in the compartment H12, and outputs a detection result to the controller 130. When the concentration of the volatile organic compound is greater than a detection threshold (e.g., 0.400 mg/m³), the controller 130 actively controls the power supply unit 110 to supply power to the fluid generating element and the ultraviolet light source.

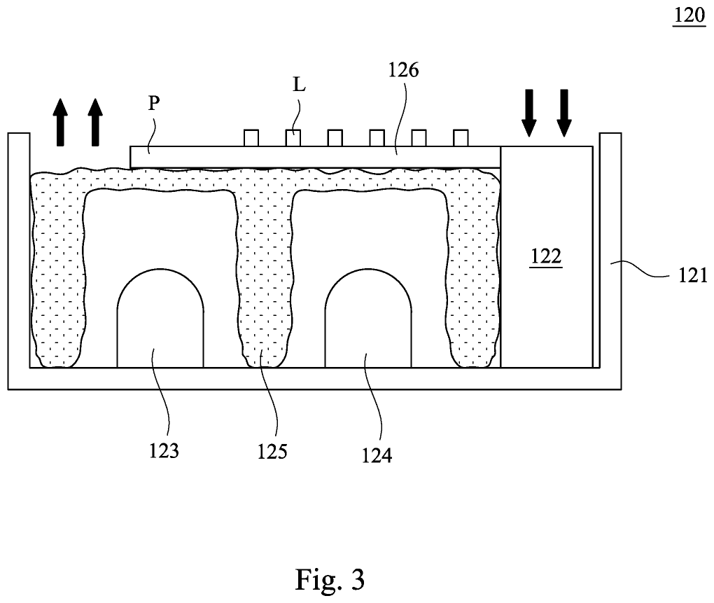

FIG. 3 is a schematic diagram of a sterilization device 120 in some embodiments of the present disclosure. The sterilization device 120 includes a housing 121, a fluid generating element 122, a first ultraviolet light source 123, a second ultraviolet light source 124, a photocatalyst element 125 and a lighting circuit 126. The fluid generating element 122, the first ultraviolet light source 123, the second ultraviolet light source 124 and the lighting circuit 126 are coupled to the power supply unit 110, and are configured to receive the power supplied by the power supply unit 110. In this embodiment, the sterilization device 120 includes only two ultraviolet light sources. However, the present disclosure is not limited to this. In other embodiments, the sterilization device 120 may include more than three of the ultraviolet light sources.

In some embodiments, the photocatalyst element 125 includes a titanium dioxide steel wool. The titanium dioxide steel wool can be implemented by using a steel wool sprayed with tiny amounts of titanium dioxide particles. A position of the photocatalyst element 125 corresponds to the fluid generating element 122 and the first ultraviolet light source 123, the second ultraviolet light source 124, so that the first ultraviolet light source 123 and the second ultraviolet light source 124 can illuminate at least a part of the photocatalyst element 125, respectively. When the ultraviolet light is illuminated on the photocatalyst element 125, the above active substances with high oxidizing ability can be formed.

In one embodiment, the photocatalyst element 125 surrounds and covers the first ultraviolet light source 123 and the second ultraviolet light source 124 to maximize the production of active substances. As shown in FIG. 3, a cross section of the photocatalyst element 125 is E-shaped, and is configured to cover the first ultraviolet light source 123 and the second ultraviolet light source 124. However, the present disclosure is not limited to this. In other embodiments, the photocatalyst element 125 can be in other shapes, and an only requirement is that the photocatalyst element 125 needs to be illuminated by the first ultraviolet light source 123 and the second ultraviolet light source 124. Similarly, the present disclosure does not limit the number of the photocatalyst element 125. In other embodiments, the sterilization device 120 includes multiple photocatalyst elements, and photocatalyst elements are arranged in an illumination range of the first ultraviolet light source 123 or the second ultraviolet light source 124, respectively.

The housing 121 is provided with an air inlet and an air outlet. Inside and outside of the sterilization device 120 are communicated to each other through the air inlet and the air outlet respectively. For example, the air inlet is adjacent to the fluid generating element 122, and the air outlet is arranged at an end of the housing 121 away from the fluid generating element 122. When the fluid generating element 122 operates, outside air is sucked by the fluid generating element 122 through the air inlet, causing the sucked air to flow through the photocatalyst element 125 illuminated by the ultraviolet light. Then, the active substances generated by the photocatalyst element 125 will flow out from the air outlet along with the airflow. The active substances are distributed in the compartment H12, so as to deodorize and inhibit bacteria to the compartment H12 and items inside the compartment H12. Alternatively, when air quality in the compartment H12 is abnormal, the fluid generating element 122 extracts the dirty air through the air inlet, and makes the dirty air interact with the active substances to purify the dirty air. Subsequently, the purified air is distributed back to the compartment H12 through the air outlet.

The lighting circuit 126 includes a circuit board P and at least one lighting element L (e.g., LED). In one embodiment, the circuit board P of the lighting circuit 126 is set above the first ultraviolet light source 123, the second ultraviolet light source 124 and the photocatalyst element 125, and completely covers the first ultraviolet light source 123, the second ultraviolet light source 124, to prevent the ultraviolet light from illuminating outside the sterilization device 120 to avoid harm to human eyes and oxidation of other materials in the compartment H12.

In addition, the vehicle sterilization system 100 further includes a built-in battery 170. The battery 170 is different from the power supply unit 110, is arranged inside the vehicle H10, and may not be exposed to the space HS. When the power supply unit 110 is removed from the vehicle H10, the controller 130 can still be drived through the battery 170. In one embodiment, the lighting circuit 126 may be coupled to the battery 170. When the seat cushion H11 does not cover the space HS of the compartment H12 (e.g., the user opens the seat cushion H11), the controller 130 controls the battery 170 to supply power to the lighting element L of the lighting circuit 126. Accordingly, even if the lighting circuit 126 is not powered by the power supply unit 110 (or the power provided by the power supply unit 110 is not enough for operating the lighting circuit 126), the lighting circuit 126 can still be driven by the battery 170, and automatically light up, so that the user can clearly see the items in the compartment H12. For example, when the vehicle H10 is shutted off, the power stored in the power supply unit 110 is exhausted, or the power supply unit 110 is removed from the vehicle H10, the lighting circuit 126 can still be powered by the battery 170 to operate.

Furthermore, when the sterilization device 120 is not powered by the power supply unit 110 (or the power provided by the power supply unit 110 is not enough), the controller 130 can further control the battery 170 to supply power to the sterilization device 120 to perform the sterilization process. In other words, when the user leaves the vehicle H10, even if the power from the power supply unit 110 is shutted off, the vehicle sterilization system 100 can still drive the sterilization device 120 through the battery 170 for sterilization.

In some embodiments, the vehicle sterilization system 100 selectively changes an operating state of the sterilization device 120 according to a positional relationship between the user and the vehicle H10. For example, a portable electronic device 200 (e.g., mobile phone) of the user and the controller 130 of the vehicle H10 can communicate with each other through any wireless communication technology (e.g., Bluetooth, Near Field Communication (NFC) or WiFi), and the controller 130 determines a distance between the vehicle H10 and the portable electronic device 200. For example, the distance is estimated based on the Received Signal Strength Indication (RSSI). In some embodiments, the operation of “estimating the distance” can be performed by the portable electronic device 200, and the portable electronic device 200 notifies the controller 130 of the estimation results.

As mentioned above, when the estimated distance is greater than a distance threshold (e.g., greater than 10 meters), the controller 130 controls the sterilization device 120 to operate in a stronger first sterilization state. The controller 130 controls the power supply unit 110 to supply power to the first ultraviolet light source 123 and the second ultraviolet light source 124, so that the first ultraviolet light source 123 and the second ultraviolet light source 124 simultaneously generate the ultraviolet light to illuminate the photocatalyst element 125. At this time, a larger amount of the active substances are generated from the photocatalyst element 125. On the other hand, when the estimated distance is less than or equal to the distance threshold (e.g., the distance is between 5-10 meters), the controller 130 controls the sterilization device 120 to operate in a weaker second sterilization state. The controller 130 controls the power supply unit 110 to supply power to only one of the first ultraviolet light source 123 and the second ultraviolet light source 124. For example, the power supply unit 110 only supplies power to the first ultraviolet light source 123, but not to the second ultraviolet light source 124. In other words, if the estimated distance between the vehicle H10 and the portable electronic device 200 is too close, the sterilization device 120 will be controlled in a weaker sterilization state to prevent potential adverse effect causing by the ultraviolet light when the user is close to the vehicle H10.

In some other embodiments, the vehicle sterilization system 100 further includes a positioning device 180 (e.g., GPS signal transceiver). The positioning device 180 is coupled to the controller 130, and is configured to obtain a current coordinate of the vehicle H10. The vehicle H10 connects to a cloud server (e.g., network map server) immediately or periodically to determine the current location and area of the vehicle H10. In some embodiments, the vehicle H10 connects to the cloud server through the portable electronic devices 200. The cloud server provides the controller 130 with information about multiple marked areas (e.g., adjacent to a medical hospital, or located in a high-risk area). When the controller 130 determines that the current coordinate is located in one of the marked areas recorded in the cloud server, the controller 130 automatically controls the power supply unit 110 to supply power to the sterilization device 120 to perform the sterilization process immediately.

As mentioned above, in some other embodiments, the sterilization process can also be performed by the cloud server remotely controlling the vehicle sterilization system 100. Specifically, after the controller 130 outputs the current coordinate of the vehicle H10 to the cloud server, the cloud server determines whether the vehicle H10 is located in one of marked areas according to a built-in map information. When the vehicle H10 is located in one marked area, the cloud server transmits a command to the controller 130, so that the controller 130 drives the sterilization device 120 to perform the sterilization process. On the contrary (the vehicle H10 is outside all of marked areas), the cloud server does not drive the sterilization device 120 to perform the sterilization process.

In some other embodiments, the vehicle H10 further switches the sterilization state of the sterilization device 120 according to the current coordinate. Specifically, after obtaining the current coordinate through the positioning device 180, the controller 130 connects to the cloud server to determine the current location and area of the vehicle H10. When the controller 130 determines that the current coordinate is located in one of marked areas recorded in the cloud server, the controller 130 drives the sterilization device 120 to operate in the stronger first sterilization state. On the contrary, when the controller 130 determines that the current coordinate is not located in all of marked areas recorded in the cloud server (i.e. the current coordinate is outside all of marked areas), the controller 130 drives the sterilization device 120 to operate in the weaker second sterilization state. Accordingly, the sterilization device 120 can perform the sterilization process in the compartment H12 more thoroughly when the vehicle H10 is parked or driven in the high-risk area of the epidemic. When the vehicle H10 stops or drives in a non-high-risk area of the epidemic, power consumption of the sterilization device 120 can be reduced.

For example, the vehicle H10 connects to the cloud server every 30 minutes to confirm whether the current coordinate is located in one of marked areas, so as to dynamically switch the sterilization state of the sterilization device 120. When the current coordinate of the vehicle H10 changes from outside the marked area to inside the marked area, the controller 130 switches the sterilization device 120 to the first sterilization state. At this time, the controller 130 can further enable the sterilization device 120 to perform the sterilization process immediately.

In addition, the controller 130 drives the positioning device 180 to obtain the current coordinate when the vehicle H10 is power on, and then determines whether the sterilization device 120 operates in the first sterilization state or the second sterilization state. Alternatively, after the vehicle H10 receives a power off command, the controller 130 can drive the positioning device 180 to obtain the current coordinates and determine the sterilization state of the sterilization device 120 during a period of time before the power supply unit 110 completely stops power supply. Accordingly, when the user powers on the vehicle H10 next time, the controller 130 can control the sterilization device 120 in the previously determined sterilization state.

As mentioned above, similarly, the sterilization state of the sterilization device 120 can be controlled remotely by the cloud server. Specifically, after the controller 130 outputs the current coordinates of the vehicle H10 to the cloud server, the cloud server determines whether the vehicle H10 is located in one of marked areas according to the built-in map information. When the vehicle H10 is located in one of marked areas, the cloud server outputs a state control signal to the controller 130, then the controller 130 drives the sterilization device 120 to operate in the first sterilization state. On the contrary, the cloud server remotely controls the sterilization device 120 to operate in the second sterilization state.

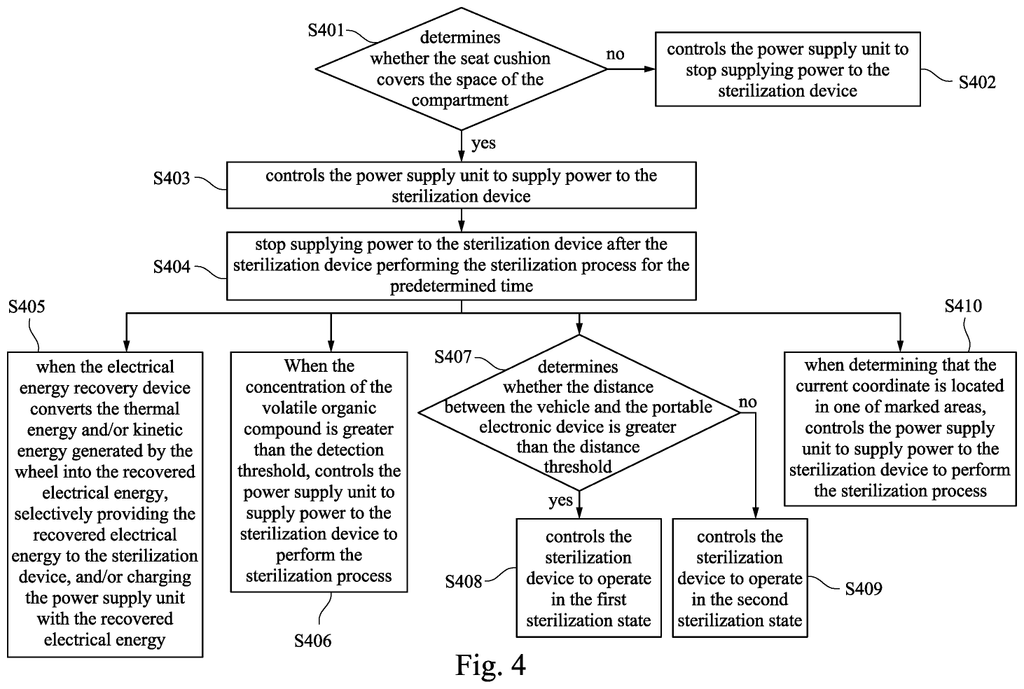

FIG. 4 is a flowchart illustrating a sterilization method applied to the vehicle sterilization system 100 in some embodiments of the present disclosure. In step S401, the controller 130 determines whether the seat cushion H11 covers the space HS of the compartment H12. In step S402, if the seat cushion H11 does not cover the space HS of the compartment H12, the controller 130 controls the power supply unit 110 to stop supplying power to the sterilization device 120. On the contrary, if the seat cushion H11 covers the space HS of the compartment H12, in step S403, the controller 130 controls the power supply unit 110 to supply power to the fluid generating element 122 and the ultraviolet light source 123, 124 of the sterilization device. In some embodiments, the power supply unit 110 can automatically supply or stop supply power to the sterilization device 120 according to the level of the received detection signal.

In step S404, when the sterilization device 120 performs the sterilization process according to the power provided by the power supply unit 110, the controller 130 controls the power supply unit 110 to stop supplying power to the sterilization device 120 after the sterilization device 120 performing the sterilization process for the predetermined time.

In addition, during the process of the vehicle H10 being powered on, the controller 130 may further perform the following steps according to different states or conditions: in step S405, when the vehicle H10 stops driving at least one wheel, and the electrical energy recovery device 150 converts the thermal energy and/or the kinetic energy generated by the wheel into the recovered electrical energy, the controller 130 selectively provides the recovered electrical energy to the sterilization device 120, and/or charges the power supply unit 110 with the recovered electrical energy.

In step S406, the controller 130 obtains the concentration of one or more specific gas molecules (e.g., volatile organic compound) in the compartment H12 through the sensor 160. When the concentration of the one or more specific gas molecules is greater than the detection threshold, the controller 130 controls the power supply unit 110 to supply power to the sterilization device 120 to perform the sterilization process.

In step S407, the controller 130 determines whether the distance between the vehicle H10 and the portable electronic device 200 is greater than the distance threshold. In step S408, when the distance between the vehicle H10 and the portable electronic device 200 is greater than the distance threshold, the controller 130 controls the sterilization device 120 to operate in the first sterilization state. In step S409, when the distance between the vehicle H10 and the portable electronic device 200 is less than or equal to the distance threshold, the controller 130 controls the sterilization device 120 to operate in the second sterilization state. As described in the previous embodiment, the number of the ultraviolet light sources enabled in the first sterilization state is greater than the number of the ultraviolet light sources enabled in the second sterilization state. In the second sterilization state, only a part of the ultraviolet light sources will be drived.

In step S410, after obtaining the current coordinate of the vehicle H10 through the positioning device 180, driving the sterilization device 120, or adjusting the sterilization state of the sterilization device 120 according to a relative relationship between the current coordinate of the vehicle H10 and the marked area. For example, when determining that the current coordinate is located in one of marked areas, the controller 130 controls the power supply unit 110 to supply power to the sterilization device 120 to perform the sterilization process.

In addition, in other embodiments, the controller 130 can also determine the distance between the current coordinate and the marked area to adjust the operating state of the sterilization device 120. For example, if the controller 130 determines that the current coordinate located in one of marked areas, or the distance between the current coordinate and a boundary of one of marked areas is within a first range (e.g., less than 10 meters, or within 5-10 meters), the controller 130 controls the sterilization device 120 to operate in the first sterilization state. On the other hand, if the controller 130 determines that the distance between the current coordinate and the boundary of one of marked areas is within a second range (e.g., 10-20 meters), the controller 130 controls the sterilization device 120 to operate in the second sterilization state.

In the above embodiments, the sterilization device 120, the controller 130, the state detection circuit 140, the electrical energy recovery device 150, the sensor 160, the battery 170 and the positioning device 180 in the vehicle sterilization system 100 can also be part of the vehicle H10. In addition, as described in the above embodiments, the “controller 130” in the foregoing steps S410-S410 is not limited to an electronic control unit in the vehicle H10, but may also be a processor or a processing circuit built in the power supply unit 110.

In the above embodiments, a power supply state between the power supply unit 110 and the sterilization device 120 corresponds to the relative relationship between the seat cushion H11 and the compartment H12. For example, when the seat cushion H11 completely covers the space HS in the compartment H12, the detection signal is at the enable level, and the controller 130 automatically controls the power supply unit 110 to supply power to the sterilization device 120. On the contrary, when the seat cushion H11 does not completely cover the space HS in the compartment H12, the detection signal is at the disable level, and the controller 130 automatically controls the power supply unit 110 to stop supplying power to the sterilization device 120. In other words, the sterilization device 120 passively receives power and is driven. In another embodiment, the vehicle sterilization system 100 may periodically determines the relative relationship between the seat cushion H11 and the compartment H12, so as to actively change the power supply state between the power supply unit 110 and the sterilization device 120.

The elements, method steps, or technical features in the foregoing embodiments may be combined with each other, and are not limited to the order of the specification description or the order of the drawings in the present disclosure.

It will be apparent to those skilled in the art that various modifications and variations can be made to the structure of the present disclosure without departing from the scope or spirit of the present disclosure. In view of the foregoing, it is intended that the present disclosure cover modifications and variations of this present disclosure provided they fall within the scope of the following claims.

What is claimed is:

1. A sterilization method for a vehicle sterilization system, comprising:
receiving a detection signal, and determining whether a seat cushion of a vehicle covers a space of a compartment according to a voltage level of the detection signal;
when the seat cushion does not completely cover the space of the compartment, controlling a power supply unit to stop supplying power; and
when the seat cushion covers the space of the compartment, controlling the power supply unit to supply power to a sterilization device.

2. The sterilization method of claim 1, wherein controlling the power supply unit to supply power to the sterilization device comprises:
driving the sterilization device for a predetermined time, and then controlling the power supply unit to stop supplying power to the sterilization device.

3. The sterilization method of claim 1, further comprising:
when the vehicle stops driving at least one wheel, converting, by an electrical energy recovery device, thermal energy or kinetic energy generated by the at least one wheel into recovered electrical energy; and
providing at least one portion of the recovered electrical energy to the sterilization device.

4. The sterilization method of claim 3, wherein providing the at least one portion of the recovered electrical energy to the sterilization device comprises:
providing a first portion of the recovered electrical energy to the sterilization device; and
using a second portion of the recovered electrical energy to charge the power supply unit.

5. The sterilization method of claim 3, further comprising:
determining a residual power ratio in the power supply unit; and
when the residual power ratio is less than a warning threshold, stopping supplying the recovered electric energy to the sterilization device, and using the recovered electric energy to charge the power supply unit.

6. The sterilization method of claim 1, further comprising:
determining, by a sensor, a concentration of a volatile organic compound in the compartment; and
when the concentration of the volatile organic compound is greater than a detection threshold, controlling the power supply unit to supply power to the sterilization device for at least a predetermined time.

7. The sterilization method of claim 1, wherein the sterilization device comprises a first ultraviolet light source and a second ultraviolet light source, and the sterilization method further comprises:
determining a distance between the vehicle and a portable electronic device, wherein the vehicle is communicatively connected to the portable electronic device;
when the distance is greater than a distance threshold, controlling the power supply unit to supply power to the first ultraviolet light source and the second ultraviolet light source; and
when the distance is less than or equal to the distance threshold, controlling the power supply unit to supply power to one of the first ultraviolet light source and the second ultraviolet light source.

8. The sterilization method of claim 1, wherein the sterilization device comprises a first ultraviolet light source and a second ultraviolet light source, and the sterilization method further comprises:
obtaining, by a positioning device, a coordinate of the vehicle;
connecting, by the vehicle or a portable electronic device, to a cloud server, wherein the vehicle is communicatively connected to the portable electronic device;
when determining the coordinate is located in a marked area recorded in the cloud server, controlling the power supply unit to supply power to the first ultraviolet light source and the second ultraviolet light source; and
when determining the coordinate is outside the marked area, controlling the power supply unit to supply power to one of the first ultraviolet light source and the second ultraviolet light source.

9. The sterilization method of claim 1, further comprising:
when the sterilization device is not powered by the power supply unit, driving the sterilization device by a battery, wherein the battery is different from the power supply unit.

10. A vehicle sterilization system, comprising:
a power supply unit arranged in a vehicle, wherein a compartment of the vehicle is provided with a seat cushion; and
a sterilization device arranged in the compartment;
wherein when the seat cushion does not completely cover a space of the compartment, the power supply unit is configured to stop supplying power;
wherein when the seat cushion covers the space of the compartment, the power supply unit is configured to supply power to the sterilization device.

11. The vehicle sterilization system of claim 10, further comprises:
a controller configured to control the power supply unit to drive the sterilization device for a predetermined time, wherein after the predetermined time, the controller is configured to control the power supply unit to stop supplying power to the sterilization device.

12. The vehicle sterilization system of claim 10, further comprises:
a controller configured to control the power supply unit to drive the sterilization device for a predetermined time; and
an electrical energy recovery device configured to convert thermal energy or kinetic energy generated by at least one wheel into recovered electrical energy when the vehicle stops driving the at least one wheel, wherein the controller is configured to provide at least one portion of the recovered electrical energy to the sterilization device.

13. The vehicle sterilization system of claim 12, wherein the controller is configured to provide a first portion of the recovered electrical energy to the sterilization device, and is configured to use a second portion of the recovered electrical energy to charge the power supply unit.

14. The vehicle sterilization system of claim 12, wherein the controller is configured to determine a residual power ratio in the power supply unit; when the residual power ratio is less than a warning threshold, the controller is configured to stop supplying the recovered electric energy to the sterilization device, and is configured to use the recovered electric energy to charge the power supply unit.

15. The vehicle sterilization system of claim 10, further comprises:
a controller configured to control the power supply unit to drive the sterilization device for a predetermined time; and
a sensor coupled to the controller, and configured to determine a concentration of a volatile organic compound in the compartment;
wherein when the concentration of the volatile organic compound is greater than a detection threshold, the controller is configured to control the power supply unit to supply power to the sterilization device for at least a predetermined time.

16. The vehicle sterilization system of claim 10, wherein the sterilization device comprises at least one fluid generating element, at least one ultraviolet light source and at least one photocatalyst element, wherein a position of the at least one photocatalyst element corresponds to the at least one ultraviolet light source.

17. The vehicle sterilization system of claim 16, wherein the at least one ultraviolet light source comprises a first ultraviolet light source and a second ultraviolet light source, and the sterilization device further comprises:
a controller configured to determine a distance between the vehicle and a portable electronic device, wherein the vehicle is communicatively connected to the portable electronic device;
wherein when the distance is greater than a distance threshold, the controller is configured to control the power supply unit to supply power to the first ultraviolet light source and the second ultraviolet light source;
wherein when the distance is less than or equal to the distance threshold, the controller is configured to control the power supply unit to supply power to one of the first ultraviolet light source and the second ultraviolet light source.

18. The vehicle sterilization system of claim 16, wherein the at least one ultraviolet light source comprises a first ultraviolet light source and a second ultraviolet light source, and the sterilization device further comprises:
a controller configured to control the power supply unit to drive the sterilization device for a predetermined time; and
a positioning device configured to obtain a coordinate of the vehicle;
wherein when the coordinate is located in a marked area recorded in a cloud server, the controller is configured to control the power supply unit to supply power to the first ultraviolet light source and the second ultraviolet light source;
wherein when the coordinate is outside the marked area, the controller is configured to control the power supply unit to supply power to one of the first ultraviolet light source and the second ultraviolet light source.

19. The vehicle sterilization system of claim 16, wherein the sterilization device further comprises:
a lighting circuit coupled to the power supply unit, and comprising a circuit board and at least one lighting element, wherein the circuit board is configured to cover the at least one ultraviolet light source to prevent a ultraviolet light generated by the at least one ultraviolet light source from being exposed to an outside of the sterilization device.

20. The vehicle sterilization system of claim 10, further comprising:
a controller configured to control the power supply unit to drive the sterilization device for a predetermined time; and
a battery coupled to the sterilization device, wherein when the sterilization device is not powered by the power supply unit, the controller is configured to drive the sterilization device by the battery.
